(19) United States
(12) Patent Application Publication
(10) Pub. No.: US 2022/0036967 A1
(43) Pub. Date: Feb. 3, 2022

(54) A METHOD TO DETERMINE AGENTS FOR PERSONALIZED USE

(71) Applicants: Thomas EFFERTH, Heidelberg (DE); Henry Johannes GRETEN, Heidelberg (DE)

(72) Inventors: Thomas EFFERTH, Heidelberg (DE); Henry Johannes GRETEN, Heidelberg (DE)

(21) Appl. No.: 17/312,249

(22) PCT Filed: Dec. 9, 2019
(86) PCT No.: PCT/EP2019/084195

§ 371 Date: Jun. 9, 2021

(30) Foreign Application Priority Data
Dec. 10, 2018 (EP) 18211231.8

(51) Int. Cl. G16B 15/30; G16C 20/50

(52) U.S. Cl. CPC G16B 15/30; G16C 20/50

(57) ABSTRACT

The present invention relates to a method for identifying one or more compounds specifically binding to a target structure of a given diseased tissue in an individual, said method comprises the determination of the binding affinity of a number of compounds to the one or more docking spaces of a mutated gene identified in the individual and identifying one or more compounds specifically binding to the mutated protein. Further, the present invention relates to a computer program comprising instructions which cause the computer to carry out several steps of the method.

The present invention relates to a method for identifying one or more compounds specifically binding to a target structure of a given diseased tissue in an individual, said method comprises the determination of the binding affinity of a number of compounds to the one or more docking spaces of a mutated gene identified in the individual and identifying one or more compounds specifically binding to the mutated protein. Further, the present invention relates to a computer program comprising instructions which cause the computer to carry out several steps of the method.

Although a variety of therapeutic strategies have been developed within the preceding decades, disorders based on diseased tissue are still often severe. In particular, a neoplasm, such as cancer, is still a life-threatening disorder. Typically, the therapeutic means used for treating neoplasms bear severe undesired side effects and are often limited in efficacy. Antineoplastic agents and irradiations typically also negatively affect healthy tissue. Despite considerable improvements in antineoplastic therapy during the past decades, the success of chemotherapy is still hampered by severe and partly life-threatening side effects that prevent to apply drug doses high enough to kill less responsive tumor cells. Further, neoplasms, such as malignant tumors, often become at least partly resistant against antineoplastic agents. As a result, resistance to anticancer drugs frequently develops. This may ultimately lead to the failure of chemotherapy with fatal outcome for many patients. Therefore, there is a desire to identify further agents and combinations thereof usable for antineoplastic treatments.

The development of further antineoplastic agents is typically highly costly and laborious. Comprehensive preclinical and clinical tests are required. Advancements in molecular diagnostics have to be complemented by novel drugs. Drug development and marketing is a time- and cost-intensive process.

The number of newly approved drugs (e.g., FDA-approved drugs) declined for decades, mainly because of their failure in clinical phase II clinical trials, despite the fact that time and expenditure on drug research and development (R&D) consistently increased during recent years [Mullard, 2011; Kola and Landis, 2004].

Conventional tumor chemotherapy is based on treatment regimens that are defined by official standard treatment guidelines. These chemotherapy protocols are based on the result of prospective, randomized, double-blind phase I-III studies. However, each tumor may behave differently. Hence, treatment success of individual patients still cannot be reliably predicted, although the statistical probability of treatment response for larger groups of patients can be estimated from the results of clinical trials. The reason is that even tumors of the same origin and histology often differ from patient to patient according to their individual biological behavior. Although an increasing number of novel targeted drugs are entering the market (e.g. small molecule inhibitors and therapeutic antibodies), many of them act in an inadequate manner without sustainable and long-lasting improvement of the tumor diseases.

Thus, it is regularly tried to identify pharmaceutically active agents for new therapeutic uses. In other words, an agent which is pharmaceutically used in another therapeutic field is further used for an antineoplastic treatment. In this context, a surprising concept emerged [Aronson, 2007].

WO 2001/035316 describes computer-based methods of drug design based on genetic polymorphisms with a focus on treating viral infections. This document does, however, not refer to diseased tissues, in particular not neoplastic tissue. The described polymorphisms may have impact on usability of antiviral agents.

WO 2003/057173 describes a method for identifying broad-spectrum inhibitors which bind to both, a known wild-type target structure and to a known variant target structure. The purpose of this document is that the inhibitor may be active throughout different variants. This method is focused on antiviral therapies. This method does, however, not allow the identifications of compounds that selectively bind to a target structure of a diseased tissue.

Existing drugs with well-known safety and pharmacokinetic profiles against certain diseases might serve as valuable drug candidates for other diseases affected by the same pathway. This phenomenon has been described as “drug repositioning” (also: drug repurposing”, “off-target use” or “off-label use”) [Ashburn and Thor, 2004].

An intriguing example of the potential of drug repositioning is thalidomide, which has been banned as barbiturate for its teratogenic effects [Vargesson, 2015]. Later on, thalidomide has been identified as effective drug against multiple myeloma [Moehler, 2012]. Drug-repurposing was described by a using a proteo-chemometric method [Dakshanamurthy et al., 2012]. This is, however, a laborious approach that requires knowledge on the chemical modifications and special computer programs focused on this approach. The artisan who wants to treat a patient still faces severe difficulties to select a suitable agent or a certain combination of agents to treat a patient in an off-target use.

In principle, this requires personalized medicine. Prior to selecting a specific off-target use, the patient has to be analyzed.

Today, this is often performed by laborious, costly and time-consuming means. In the past, it has been attempted to identify the molecular basis of drug resistance and to predict, a priori, whether or not an individual tumor would respond to standard drug therapy [Volm and Efferth, 2015]. The aim was to adapt treatment in the clinic according to the individual drug sensitivity profile of tumors predicted beforehand [Walther and Sklar, 2011]. The challenge of this concept is to delineate individual and efficient treatment strategies, which are superior to traditional concepts of standardized tumor treatment [Schmidt and Efferth, 2016; Efferth et al., 2017; Mbaveng et al., 2017; Hientz et al., 2017]. The hope was that the emerging new technologies based on the molecular architecture of individual tumor genomes will help to generate novel anticancer drugs for the market.

A computer-based approach that describes the binding of the given single plant-derived non-drug substance oridonin which is was found to have an effect on cells and which is suspected to bind to given cellular target structures was described [Kadioglu et al., 2018]. The purpose was to provide further evidence for the usability of oridonin in tumor cells. Herein, the binding of this specific substance to target structures was simulated. Such computer-based approach is, however, not suitable for drug-repurposing. Neither a selection of compounds nor an approved drug compound is used.

As indicated above, personalized medicine faces several challenges. In particular drug-repurposing is technically challenging. Often, it requires rather complex and laborious analytical steps. Further, in a final step, the doctor has to make a selection merely based on a scientifically often unfounded experience. Accordingly, there is still an unmet disease for an improved method for identifying one or more compounds specifically binding to a target structure of a given diseased tissue.

Surprisingly, it has been found that compounds specifically binding to a given diseased tissue such as a given diseased tissue, in particular a given neoplasm, can be effectively and easily selected by a method which comprises the determination of the binding affinity of a number of compounds to the one or more docking spaces of a mutated gene identified in the diseased tissue by means of molecular docketing. The present invention relates to a method which allows the prediction of drug effects according to the individual mutations and mutational patterns of patients such as in cancer and other genetic diseases.

A first aspect relates to a method for identifying one or more compounds specifically binding to a target structure of a given diseased tissue, said method comprising the following:
(i) identifying a mutated gene in the transcriptome of said diseased tissue and identifying at least one mutation comprised in said mutated gene;
(ii) providing a three-dimensional (3D) structure of a wild-type or homolog protein expressed by a wild-type or homolog gene corresponding to the mutated gene identified in step (i);
(iii) determining a 3D structure of a mutated protein which is the expression product of the mutated gene identified in step (i) or one or more docking spaces thereof, comprising:
   (a) adapting the amino acid sequence of the 3D structure of the wild-type or homolog protein of step (ii) to the expression product of the mutated gene identified in step (i) and defining one or more docking spaces of the obtained 3D structure of mutated protein, or
   (b) defining one or more docking spaces of the 3D structure of the wild-type or homolog protein of step (ii) and adapting the amino acid sequence of said one or more docking spaces to the expression product of the mutated gene identified in step (i);
(iv) providing 3D structures of a selection of compounds and fitting each 3D structure of each compound with the one or more docking spaces of step (iii);
(v) determining the binding affinity of each compound to the one or more docking spaces; and
(vi) identifying one or more compounds specifically binding to the mutated protein.

The method of the present invention relates to an individual allocation of pharmaceuticals to diseased tissues, in particular neoplastic diseases such as cancer, and other genetic diseases based on patient-specific mutation profiles.

In a preferred embodiment, the mutated gene, the mutated protein, or a combination thereof is associated with a neoplasm. Accordingly, in a preferred embodiment, the mutated gene is associated with the onset or progression of a neoplasm. Additionally or alternatively, the mutated protein is associated with the onset or progression of a neoplasm. In a preferred embodiment, the mutated gene and the mutated protein are associated with the onset or progression of a neoplasm, in particular a tumor.

In a preferred embodiment, the present invention refers to a method that may be based on the determination of genome-wide mutations in transcribed genes and the identification of drugs that act against specific mutations in each individual. In a preferred embodiment, the present invention focuses on known drugs that are in use for other diseases and that can be repurposed for individualized tumor therapy (also designated as “drug repositioning”, “drug repurposing” or “off-target use”).

Preferably, the method of the present invention is an in vitro method conducted outside of the individual's body. In a preferred embodiment, the method of the present invention is a computer-implemented method. In other words, some or all calculations of the method are conducted in a computer-assisted manner. Optionally, the computer-assisted manner may include the conduction of one or more procedural steps on a supercomputer. An example for a supercomputer which is applicable is MOGON II (Mainz, Germany).

In a preferred embodiment, one, some or all of steps (ii) to (vi) are partly or completely conducted by a computer.

In a preferred embodiment, at least one of steps (ii), (iii), (iv), (v) and/or (vi) is conducted in a computer-assisted manner. In a preferred embodiment, at least two of steps selected from steps (ii), (iii), (iv), (v) and/or (vi) are conducted in a computer-assisted manner. In a preferred embodiment, at least steps (ii) and (iii), at least steps (ii) and (iv), at least steps (ii) and (v), at least steps (ii) and (vi), at least steps (iii) and (iv), at least steps (iii) and (v), at least steps (iii) and (vi), at least steps (iv) and (v), at least steps (iv) and (vi), or at least steps (v) and (vi) and conducted in a computer-assisted manner. In a preferred embodiment, at least three of steps selected from steps (ii), (iii), (iv), (v) and/or (vi) are conducted in a computer-assisted manner. In a preferred embodiment, at least steps (iii), (iv) and (v), at least steps (iii), (iv) and (vi), at least steps (ii), (iv) and (v), at least steps (ii), (iv) and (vi), at least steps (ii), (iii) and (v), at least steps (ii), (iii) and (vi), at least steps (ii), (iii) and (iv), or at least steps (ii), (iii) and (vi) are conducted in a computer-assisted manner. In a preferred embodiment, at least four of steps selected from steps (ii), (iii), (iv), (v) and/or (vi) are conducted in a computer-assisted manner. In a preferred embodiment, at least steps (iii)-(vi), at least steps (ii), (iv), (v) and (vi), at least steps (ii), (iii), (v) and (vi), at least steps (ii), (iii), (iv) and (vi), at least steps (ii), (iii), (iv) and (v) are conducted in a computer-assisted manner. In a preferred embodiment, steps (ii)-(vi) are conducted in a computer-assisted manner.

In a highly preferred embodiment, at least steps (ii)-(v) are conducted in a computer-assisted manner.

As used herein, the term “diseased tissue” may be understood in the broadest sense as any tissue that bears diseased properties such as, e.g., excessive growth, unhealthy secretion of extracellular matrix or secretes. In a preferred embodiment, such diseased tissue bears at least one mutation. In a preferred embodiment, the diseased tissue is diseased dues to at least one mutation in its genome. Such diseased tissue can also be designated as “genetic disease” in the broadest sense. These genetic diseases do not necessarily have to be hereditary diseases, but may also be diseases acquired by one or more postnatal mutations. In a preferred embodiment, the diseased tissue is characterized in that it bears one or more a mutations, in particular one or more mutations associated with the disease state of the diseased tissue, in particular associated with a neoplasm, thus, preferably the onset or progression of a neoplasm. In a preferred embodiment, the diseased tissue is characterized in that it bears one or more a mutations associated with a tumor, thus, preferably the onset or progression of a tumor. In other words, the mutation is preferably a driver mutation. In contrast to a driver mutation, a passenger mutation does not affect the disease state of interest of the diseased tissue.

As used in the context of the present invention, the term “associated with the disease state” may be understood in the broadest sense as (potentially) being a reason/cause of the tissue as being diseased tissue. In other words, the term “associated with the disease state” may be preferably also understood interchangeably with “causing the diseased state” or “affecting the health state”. It may be the sole reason/cause or may be one belong other reasons/causes. Preferably, the association with the disease state means that the tissue would not be as diseased if the factor associated with the disease state would not be present.

In a preferred embodiment, the diseased tissue is identified as being a genetic variant, in particular as having one or more mutations, one or more (different) alleles, one or more polymorphisms, or combinations of two or more thereof, in comparison to the corresponding healthy tissue. In a preferred embodiment, the diseased tissue is identified as being one or more mutations in comparison to the corresponding healthy tissue. In a preferred embodiment, the diseased tissue is identified as being one or more mutations associated with the disease state of the diseased tissue, in particular associated with the onset or progression of a neoplasia, in comparison to the corresponding healthy tissue. As used in this context, the corresponding healthy tissue may be tissue of the same tissue type origin as the diseased tissue. The corresponding healthy tissue may originate from the same of another individual of the same species as the diseased tissue.

The diseased tissue may be spread all over the individual's body or may be a lesion of diseased tissue. In other words, (essentially) all body cells may bear a certain mutation or only those of a specific lesion may bear a certain mutation. In a preferred embodiment, the diseased tissue is a lesion of diseased tissue. It will be understood that the diseased tissue typically originates from an individual of interest. Thus, it is typically not a pathogen, thus is not a virus, is not a bacterium, is not a fungal, and is not a pathogenic protozoon. This will also be understood as single cell organisms do not form a tissue.

As used herein, the term “mutation” may be understood in the broadest sense as any alteration of the nucleotide sequence of a nucleic acid (i.e., RNA or desoxyribonucleic acid (DNA)). Preferably, the mutation also results in an altered amino acid sequence of a protein that results from the translation of the mutated gene. Preferably, a mutation is associated with the disease state of the diseased tissue. In other words, the mutation is preferably a driver mutation. Preferably, a mutation is associated with a neoplasm. It may be associated with the onset or progression of a neoplasm.

As used herein, the term “mutated gene” may be understood in the broadest sense as a gene that bears a permanent mutation of the nucleotide sequence of the genome of the cells of the diseased tissue, in particular neoplastic cells (i.e., the cells forming a neoplasm).

As used herein, the term “transcriptome” may be understood in the broadest sense as the set of all ribonucleic acid (RNA) molecules in the diseased, in particular neoplastic, cells, in particular the messenger RNA (mRNA) molecules in the diseased, in particular neoplastic, cells. It provides information on which part of the genome, in particular the exome, is transcribed into mRNA. Typically, the transcriptome also indicates which proteins are produced in the diseased, in particular neoplastic, cells, and, thus, gives hints on the proteome. The transcriptome may also reflect pre- and/or post-transcriptional splicing. Alternatively, mRNA may also be non-coding RNA and/or epigenetically changed DNA sequences.

As used herein, an “allele” may be understood as a variant form of a given gene. An allele may or may not be associated with a with the disease state of the diseased tissue.

As used herein, a “polymorphism” may be understood as the occurrence of two or more different genetic forms, optionally also leading to different phenotypes, in the population of a species. A polymorphism may or may not be associated with a with the disease state of the diseased tissue. A polymorphism may also be a single-nucleotide polymorphism (SNP) associated with a substitution of a single nucleotide that occurs at a specific position in the genome. Preferably, such SNP variation is present at a level of more than 1% in the population.

In a preferred embodiment, the diseased tissue is a neoplasm.

As used herein, the term “neoplasm” may be understood in the broadest sense as any abnormal and excessive growth of tissue. A neoplasm may be a benign or a malignant neoplasm. In a preferred embodiment, a neoplasm is a malignant neoplasm. In a preferred embodiment, a neoplasm is a (cancerous) tumor, in other words, the individual suffers from cancer. A malignant neoplasm may be a primary tumor, a secondary or tertiary tumor and/or may be a metastasis. It will be understood that an individual may optionally also bear more than one neoplasms of the same type and/or different types.

As used herein, the term “individual” may be understood in the broadest sense as any animal or human subject that can bear diseased tissue, in particular a neoplasm. In a preferred embodiment, an individual is a mammal including human such as, e.g., a human being, a domestic mammal (e.g., a dog, a cat, a horse, a camel, cattle, a sheep, a goat, a donkey, etc.) or a wild animal. In a highly preferred embodiment, an individual is a human. An individual may also be designated as “patient” or “subject”. Typically, the individual bears at least one lesions of diseased tissue, in particular at least one neoplasm. The individual may or may not suffer from a lesion of diseased tissue such as neoplasm. The diseased tissue such as a neoplasm may optionally also be such not causing any symptoms.

In the context of the present invention, the terms “protein” and “polypeptide” may be understood interchangeably in the broadest sense as a compound mainly composed of natural amino acid moieties consecutively conjugated with another via amide bonds. It will be understood that a protein in the sense of the present invention may or may not be subjected to one or more posttranslational modification(s) and/or be conjugated with one or more non-amino acid moiety/moieties. The termini of the protein may, optionally, be capped by any means known in the art, such as, e.g., amidation, acetylation, methylation, acylation. Posttranslational modifications are well-known in the art and may be but may not be limited to lipidation, phosphorylation, sulfatation, glycosylation, truncation, oxidation, reduction, decarboxylation, acetylation, amidation, deamidation, disulfide bond formation, amino acid addition, cofactor addition (e.g., biotinylation, heme addition, eicosanoid addition, steroid addition) and complexation of metal ions, non-metal ions, peptides or small molecules and addition of iron-sulphide clusters. Moreover, optionally, co-factors such as, e.g., cyclic guanidinium monophosphate (cGMP), ATP, ADP, NAD+, NADH+H+, NADP+, NADPH+H+, metal ions, anions, lipids, etc. may be bound to the protein, irrespective on the biological influence of these co-factors.

The step (i) of identifying a mutated gene and the at least one mutation may performed by any means. The sub-step of identifying a mutated gene in the transcriptome of said diseased tissue, in particular a neoplasm, and the sub-step of identifying at least one mutation comprised in said mutated gene may optionally be performed concomitantly in a single step. In other words, a mutation may be identified in a gene. This gene is then also identified as mutated gene.

In a preferred embodiment, the step (i) of identifying a mutated gene and the at least one mutation comprises:
(a) providing a sample from the diseased tissue containing mRNA;
(b) optionally isolating and/or purifying the mRNA;
(c) optionally generating cDNA from the mRNA by means of polymerase chain reaction; and
(d) identifying at least one mutation by means of at least one step selected from the group consisting of:
   sequencing the mRNA and/or the cDNA;
   hybridizing the mRNA and/or the cDNA with a chip containing a variety of single-stranded nucleotides embracing mutated and non-mutated sequences; and
   conducting polymerase chain reaction with a number of primers including those specific for a particular mutation.

As an alternative to coding mRNA, also non-coding RNA and epigenetically changed DNA sequences may be used, as well as proteins, peptides, lipids, and all other metabolic chemical substances

The step of providing a sample from the diseased tissue, in particular neoplasm, containing mRNA may be performed by any means. Typically, the sample is obtained from a diseased tissue, in particular neoplasm. Optionally, a test sample may be taken up directly after removal into an RNA stabilization solution. It may be a stored sample or a sample previously dissected from the diseased tissue, in particular neoplasm. As indicated above, preferably, all steps (i)-(vi) of the method of the present invention are conducted in vitro method, i.e., conducted outside of the individual's body. The person skilled in the art is well aware of isolating and purifying mRNA. Complementary DNA (cDNA) may be generated by generally known means from the isolated and, optionally, purified mRNA by means of reverse transcriptase, optionally combined with polymerase chain reaction (PCR). The person skilled in the art commonly knows such methods.

In a preferred embodiment, mRNA is isolated and, optionally, purified from total RNA. Thus, in a first step, total RNA isolation may be performed, for instance, with a column-based extraction procedure to obtain pure RNA without DNA digestion. Genomic DNA may be selectively removed by a specific lysis step. Such method is applicable for cells, solid tissues, blood and other body fluids. Total RNA quality and quantity may be evaluated by a microfluidics-based platform. After loading, the sample may migrate through micro-channels to electrophoretically separate the sample components. The fluorescent probe may intercalate into RNA strands and the fluorescence may be recorded. Poly A+RNA may be isolated, fractionated and double-stranded cDNA may be synthesized. If new RNA isolation methods appear with time, they may be implemented in the entire protocol or will replace the current ones. This is exemplified further below in the experimental section. The quality of the RNA may be tested and a threshold set may be set for an RNA integrity score such as, e.g., 3 or higher, 4 or higher, 5 or higher, 6 or higher such as, e.g., of 6.8 or higher. To exclude ribosomal RNA sequences from further analysis, the RNA may optionally be hybridized with eukaryotic ribosomal RNA biotin-labeled oligonucleotide probes to deplete ribosomal RNA from total RNA. For the preparation of poly A+RNA, streptavidin-coated magnetic beads coupled with oligo-dT may optionally be used.

Few micrograms, such as, e.g., between one and ten, such as e.g., (approximately) five micrograms total RNA may be mixed with beads and RNA purification beads and incubated. After incubation for few minutes (e.g., up to 30 min, e.g., 5-10 min), the beads may be pelleted and the supernatant can be discarded. The beads may optionally be washed. The beads may be resuspended in elution buffer to elute RNA from the beads. Then, a binding process with binding buffer may take place again. The RNA bead mix may be eluted again and the RNA may be fragmented by heat treatment at approximately 50 to 90° C. or 60 to 75° C., for few minutes. The elution and prime mix may contain hexamers with random sequences and reverse transcriptase and may be used to start cDNA synthesis from the RNA templates the supernatant may be transferred to the master mix and put into a PCR plate with the barcode sequence. If the thermal cycling is finished, the RNA strand may be removed and substituted by a second cDNA strand. Using specific beads, double-stranded cDNA may optionally be separated from RNA and the reaction mix. Overhanging strand ends from fragmentation will finally be digested by 3′-5′ exonuclease to blunt ends. 5′ overhangs may be filled to blunt ends by polymerase.

The mutated protein may be any mutation known in the art. In a preferred embodiment, a mutation is not a frame-shift mutation. In a preferred embodiment, a mutation essentially maintains the 3D structure of the whole mutated protein in comparison to the corresponding non-mutated (wildtype) protein. In a preferred embodiment, and the mutated protein differs from the non-mutated protein by:
(A) a single amino acid moiety (point mutation) or two, three or more amino acid moieties;
(B) a truncation of one, two, three , four five, up to ten, up to 20, up to 50, up to 100 or even more than 100 terminal amino acid moieties;
(C) a truncation of one, two, three , four five, up to ten, up to 20, up to 50, up to 100 or even more than 100 terminal amino acid moieties;
(D) an elongation by one, two, three , four five, up to ten, up to 20, up to 50, up to 100 or even more than 100 terminal amino acid moieties; or
(E) a combination of two or more thereof.

In a preferred embodiment, the mutation is a point mutation and the mutated protein differs from the non-mutated protein by a single amino acid moiety only and each docking space embraces the different single amino acid moiety.

The person skilled in the art is aware of a number of means and procedural steps for identifying a mutation, in particular a point mutation. A mutation may be determined by any means. For instance, it may be performed by RNA-sequencing. Then, end-repaired, A-tailed and adaptor-ligated cDNA may be PCR-amplified, such as, e.g., by 5 to 20, e.g., 10 cycles. The library may be sequenced in paired-end mode (2×100 bp) using commercial RNA sequencing systems. Optionally, the resulting sequences may be aligned to a reference genome. Discrepencies concerning point mutations, deletions amplifications insertions etc. may be recorded. Optionally normalized RNA expressions may be quantified using the RPKM measure. RPKM values for transcripts and the ratios of transcripts may be taken into consideration to calculate the overall RPKM value for each gene. This is exemplified further below in the experimental section.

In step (ii) of the method of the present invention, the wild-type or homolog gene corresponding to the mutated gene identified in step (i) is identified. In other words, the wild-type or homolog counterpart gene of the mutated gene is identified. In a preferred embodiment, the wild-type counterpart corresponding to the mutated gene identified in step (i) is identified. A wild-type gene may be understood in the broadest sense as gene typically occurring in the respective healthy, i.e., non-diseased (e.g., non-neoplastic) tissue of the individual. As used throughout the present invention, the term “homologue” in the context of a gene may be understood in the broadest sense as a corresponding gene, preferably a corresponding wild-type gene, of another species.

This species is preferably rather closely related. When the individual is a human, for instance, the homologue gene is preferably a gene from another mammal. Preferably, a homologue as used herein is also a wild-type gene or a mutant gene with known three-dimensional (3D) structure.

In an optional embodiment of the present invention, the diseased tissue is compared with comparable healthy tissue. Then, the comparable healthy tissue is preferably obtained from the same individual (as the diseased tissue), more preferably wherein the diseased tissue is neoplastic tissue and the comparable healthy tissue is corresponding non-neoplastic tissue of the same individual (as the diseased tissue). Alternatively, the comparable healthy tissue may be obtained from another individual (than the diseased tissue) of the same species, wherein the diseased tissue may optionally and preferably be a neoplasm and the comparable healthy tissue is the corresponding non-neoplastic tissue of another individual (than the diseased tissue). The comparison between the diseased tissue with comparable healthy tissue may preferably be comparing a target structure characterized in that it is selected from the group consisting of one or more individual mutations, one or more (different) alleles, one or more polymorphisms, or combinations of two or more thereof, in particular wherein each target structure may be associated with a neoplasm such as, e.g., a tumor.

In a preferred embodiment, the diseased tissue bears one or more genetic variations selected from the group consisting of one or more mutations, one or more (different) alleles, one or more polymorphisms, or combinations of two or more thereof, in comparison to the corresponding healthy tissue,

In a preferred embodiment, the diseased tissue bears one or more mutations associated with the disease state of the diseased tissue (also designatable as driver mutations) in comparison to the corresponding healthy tissue.

The comparison between the diseased tissue with comparable healthy tissue may be comparing the binding of one or more compounds specifically binding to one or more target structures. The comparison between the diseased tissue with comparable healthy tissue may preferably comparing the specific binding of the one or more compounds to one or more target structures of a given diseased tissue with the binding of said one or more compounds to target structures which are the counterparts in healthy tissue of the one or more target structures of the given diseased tissue.

For example, the mutated gene may be selected from the group consisting of:

| Mutated genes | Type of mutation | Amino acid exchange |
|---|---|---|
| ABCA1 | missense variant | V825I |
| ABCB1 | missense variant | S893A |
| AKR1C3 | missense variant | E77G |
| ANKRD27 | missense variant | P761G |
| ANXA5 | missense variant | I81T |
| APOBEC3B | missense variant | K146T |
| ATP7B | missense variant | S406A und V1140A |
| CASP8 | missense variant | K344H |
| HLA-B | missense variant | H140S und I218V |
| NQO1 | missense variant | P187S |
| PARP1 | missense variant | V762A |
| TLR1 | missense variant | N248S |

From the mutated gene identified in step (i), the person skilled in the art can easily and directly deduce the respective protein. This protein is the expression product of the gene. Many 3D structures of wild-type proteins are known from databases. Such 3D structure of the wild-type or homolog gene corresponding to the mutated gene identified in step (i) is provided. The 3D structure may be any 3D structure.

In a preferred embodiment, the 3D structure of the wild-type or homolog protein of step (ii) is a crystal structure, a 3D NMR structure or a calculated hypothetical three-dimensional structure and is, optionally, obtained from a structure database.

It may be examined whether three-dimensional protein crystal structures are available encoded by genes found to be mutated from the comparison of the mutational profile obtained from RNA sequencing with protein crystal structures of the correspondingly affected proteins. In cases, where isoforms or splicing variants of target proteins are available, several homology models may optionally be prepared in parallel. Information on alterations in helices, disulfide bridges secondary loop structures, distortions of beta-sheets etc. may change the protein conformation and therefore may alter the binding properties of drugs. In selected cases, sequence alignments of proteins from different species may be performed, because the interspecies comparison may give information of interest about commonly conserved and unique sequence motifs, key amino acid positions in the pharmacophore domains, identical location of helix bending residues etc. Furthermore, co-crystallization of target proteins with other binding proteins, small molecules, antibodies, peptides etc., may optionally be used since they may not only stabilize the protein of interest, but also change its conformation from an inactive in to an active state and vice versa. In addition or alternatively, electrostatic potential maps may be calculated to determine hot spots of electron density that may interfere with binding properties of affected amino acid residues. This information may be of interest to find the most appropriate small molecule inhibitor drugs.

The method of the present invention may be conducted on at least one (high performance) computer running on an operation system such as, e.g., Linux etc. to meet the requirements of the multi-stage process of protein modeling. Either the crystallography-based structures of human target proteins or corresponding crystal structures from other species may be used for homology modeling.

A computational docking approach may be used to predict the free binding energy (kcal/mol) and pKi value (μM) of a ligand (e.g., drug) to its receptor (e.g., target protein). Force field potentials may be used to calculate the free binding energy at a given binding conformation, and the conformational space between ligand and receptor may be estimated.

Computer-assisted (e.g., internet-based or locally stored) databases for protein crystal structures may be searched for the availability of three-dimensional (3D) structures that could be used as templates to create models of patient-specific mutations. In cases, where crystal structures of the individual species (e.g., human) proteins are not available, corresponding protein structures from other species (homologs) may serve a template to generate human protein homology models.

As described before, homology modeling may be based on the creation of three-dimensional models of proteins with known amino acid sequence, but unknown crystal structure.

A precondition for homology modeling may be the existence of a crystal structure of a related protein. With an available crystal structure (e.g. a wild-type protein), the sequence of the known (wild-type) protein may be aligned to the protein with the still unknown 3D-structure (e.g. the mutant counterpart of the wild-type protein). Based on the known crystal structure of the wild-type protein, a hypothetical 3D-structure of the corresponding mutant protein may be calculated. It will be understood that such homology model may be as better as more conserved are the amino acid sequences of known and unknown proteins. As a first step, the protein sequence may be downloaded from a corresponding website, (e.g. UniProt) in FASTA format. Then, the known 3D structure of the related protein, which should serve as template, may be downloaded and both protein sequences may be compared using BLAST (Basic Local Alignment Search Tool) and ClustalW2.

Step (iii) of determining a 3D structure of a mutated protein may be performed by any means. Preferably, based on the 3D structure of the corresponding wild-type protein or the homolog, the aforementioned structure is altered by the altered amino acid moieties.

In a preferred embodiment, this is performed by the generation of mutation-specific protein homology models that resembling the mutated genes in individual diseased tissues (diseased tissue lesions), in particular neoplasms. Either the 3D structures of wild-type proteins or homologous 3D structures from other species may be used for homology modeling. 3D structures or homology models of wild-type proteins may then be modified by insertion of the amino acid exchanges delineated from RNA-sequencing of the specific diseased tissue, in particular neoplasm. The subsequent homology models of mutated proteins may be created using the alignment file with appropriate alignment programs. The Swiss-MODEL structure assessment tool may be then used to select the best homology model for molecular docking. Model evaluation may be done with the help several tools (Anolea, GROMOS, QMEAN, DFIRE etc.). In a cellular environment, proteins typically exist in a hydrated form. Therefore, hydrogens may be added to Asn and Gln residues. This is exemplified further below in the experimental section.

Step (iv) of providing 3D structures of a selection of compounds and fitting each 3D structure of each compound with the one or more docking spaces of step (iii) may be performed by any means. This step may also be designated as “bioinformatic screening” or “virtual drug screening”.

Those skilled in the art will directly and unambiguously understand that the term “selection of compounds” may typically be understood in the broadest sense interchangeably with terms like “multitude of compounds” or “variety of compounds” as a set of more than one compound, in other words, more than one type of compounds. Thus, a selection of compounds typically comprises at least two (different) compounds. It may also be a library of compounds (also designatable as compound library). It will be understood that, in the context of the method of the present invention, the selection of compounds does not necessarily mean a physically existing composition wherein the different compounds are mixed with another. Rather, each 3D structure of each compound of the selection of compounds may preferably be fitted individually with the one or more docking spaces of step (iii) (cf. step (iv) of the present invention).

In a preferred embodiment, the selection of compounds used in step (iv) comprises at least five compounds, at least ten compounds, at least 25 compounds, at least 50 compounds, at least 100 compounds, at least 250 compounds, at least 500 compounds, or at least 1000 compounds. According to step (iv), the 3D structure of each of these compounds is provided and each 3D structure of each compound is fitted with the one or more docking spaces of step (iii).

The compound of which 3D structures are provided in step (iv) may have any molecular weight. In a preferred embodiment, at least one of the compounds of which 3D structures are provided in step (iv) is a small molecule having a molecular weight of not more than 5000 Da, not more than 2000 Da, not more than 1000 Da or not more than 750 Da.

The compound of which 3D structures are provided in step (iv) may be an improved antineoplastic agent or may not be an approved as an antineoplastic agent. It may or may not have known pharmacokinetic properties. In a preferred embodiment, the compound of which 3D structures are provided in step (iv) is not an approved antineoplastic agent but has known pharmacokinetic properties.

In a preferred embodiment, the compound is approved for one or more pharmaceutical purposes other than antineoplastic activity.

In a preferred embodiment, the compound is a small molecule having a molecular weight of not more than 1000 Da or not more than 2000 Da and is approved for one or more pharmaceutical purposes other than antineoplastic activity. In a preferred embodiment, the method is a bioinformatic screening method. Preferably, a library of several compounds is tested (also: screened). Then, the compound may also be designated as a candidate compound. For instance, a library of several compounds, several dozens of compounds, several hundreds of compounds or even more than 1000 compounds (e.g., (FDA-(approved drugs), may be used to investigate the binding of drug to the mutation-specific protein homology models by means of specific virtual drug screening programs. Preferably, the variety of compounds comprises compounds not approved as antineoplastic compounds (not approved as anticancer drugs). The idea is that drugs frequently do not act in a mono-specific manner, but have broader activity spectra. Therefore, drugs for a specific disease indication may also inhibit related mutated proteins as in diseased tissues, in particular neoplasms. These inhibitory drugs may be identified by bioinformatical calculation of drug-protein binding affinities. With this approach, approved drugs can be used off-label to treat individual's diseased tissues, in particular neoplasms, according to their individual mutations.

The advantage of focusing on repurposing of FDA-approved drugs may be their (already demonstrated) biological activity and acceptable safety/toxicity profiles. This is typically not the case for chemical libraries of compounds not approved for use in human subjects.

Step (v) of determining the binding affinity of each compound to the one or more docking spaces may be performed by any means. Several algorithms may be used to identify the best binding drugs with independent techniques. In a preferred example, the 10 top-ranked out of a variety of compounds with highest affinities may be selected.

A so-called “unbiased” method may be used, where the program begins at a random position to explore the protein surface for optimal binding of a ligand. In a first screening, a program may be used that calculates the binding of a flexible chemical drug to a rigid protein surface (“rigid docking”). When interesting drugs are identified by this approach, a docking program may be applied that allows to calculate the docking of flexible drug structures to flexible protein surfaces (“flexible docking”).

Homology-modeled mutant patient-specific proteins may be set as rigid receptor molecules. The prepared output files may indicate information on atomic partial changes, torsion degrees of freedom and different atom types will be added, e.g. aliphatic and aromatic carbon or polar atoms forming hydrogen bonds such as, e.g., in PDQT format. In cases, where target proteins contain known pharmacophore sites, grids around selected amino acid residues of that pharmacophore to calculate drug binding (defined docking approach) may be used. In those cases, where no drug-binding site of a target protein is known, interaction energies for the whole protein (blind docking approach) may be first calculated. The region showing with the highest binding affinity may then be used to set a grid and a defined docking will follow as a second step. A grid box may then be constructed to define docking spaces.

For each grid point, the pairwise interaction energy between ligand and receptor may be added up over all protein atoms and saved. Separate calculations may be performed for each atom in the ligand concerning their binding energy, including electrostatics, hydrogen binding energy, dispersion/repulsion, desolvation and torsional entropy as critical parameters.

“Affinity grids” based on force field potentials may be considered for van der Waals and electrostatic interactions as well as “energy grids”, where the ligand is used in full atomic detail, while the ligand binding domain is simplified.

The dimensions of the grid box may be set around the entire protein (blind docking approach) or around defined pharmacophore sites (defined docking approach) in a manner that the ligand could freely move and rotate in the docking space. The grid box may consist of, for instance, at least 25, between 50 and 10000, between 60 and 1000, between 70 and 500, between 80 and 300, between 90 and 200 or between 100 and 150 grid points in all three dimensions (X, Y and Z axes) separated by a distance of for instance 1 between each one. As an example 126 grid points may be used. This is exemplified further below in the experimental section.

Energies at each grid point may then be evaluated for each atom type present in the ligand, and the values were used to predict the energy of a particular ligand configuration. Three independent docking calculations may be conducted, with at least 100, at least 1000, at least 10,000, at least 100,000, at least 1,000,000 or at least 10,000,000 energy evaluations. Three independent docking calculations may be conducted, with at least 2, at least 5, at least 10, at least 50, at least 100 or at least 200 runs. A Lamarckian Genetic Algorithm may be used. In a preferred embodiment, determining the binding affinity of each compound to the one or more docking spaces includes using Lamarckian Genetic Algorithm. As an example, 25,000,000 energy evaluations and 250 runs by using the Lamarckian Genetic Algorithm may be used. This is exemplified further below in the experimental section.

“Run” typically is a single docking process initiated by a UNIX-based command and controlled by a single docking parameter file. The computational docking approach can usually reveal standard deviations of up to 2 kcal/mol. Therefore, single calculations are often not sufficient. In a preferred embodiment, at least three independent docking campaigns with 25,000,000 energy evaluations and 250 runs are performed to yield reliably stable results.

The corresponding binding energies and the number of conformations in each cluster may be attained from the docking log files (dig). The corresponding lowest binding energies (LBE) may be obtained from the docking log files (dig), and mean values (optionally accompanied by standard deviations, ±SD) may be calculated. The docking results may be visualized to prove the correct binding of the drugs to the relevant drug-binding sites of the mutated tumor proteins.

Two-dimensional chemical structures may be converted to three-dimensional ones using appropriate software programs. The energy of the compound may be minimized and the new structure may be saved on the computer (e.g., as mol file). For subsequent molecular docking, the files of the ligands may be prepared in another format suitible for further processing (e.g. in pdbqt format, gpf, glg, or dpf file format). Then, the script for running the docking may be prepared. Each calculation may have maximum runtime of several hours to several days (e.g., between 2 h and 30 days, between 5 h and 20 days, between 12 h and ten days, between one and nine days, between two and eight days, between three and seven days, e.g., approximately five days (=7200 min). Each calculation may be started using the script. The results of the running jobs may be saved (e.g., in the directory of the ligand). After finalizing the jobs, the results may be optionally copied to (personal) computers. For docking campaigns of a higher number of ligands, a node-long script may be used.

FDA-approved drugs identified by the procedure described above to bind to mutated proteins may also be docked to crystal structures or homology models of the corresponding wild-type proteins.

In those cases, where the co-crystallized structure of a known ligand and its receptor is typically available, docking may be performed between the newly identified ligand by the procedure described above and its receptor b using the co-crystallized conformation as template for docking. Despite all computational predictions, all docking results are visually inspected for plausibility to exclude apparent false positive his and to increase the success rate of identified FDA-approved drugs for repurposing in cancer therapy.

Furthermore, it may be taken into account that a drug that has been identified to bind to a given target protein found in the genome of the diseased tissue, in particular neoplastic tissue, of an individual might not only bind to this protein but also to several others. Binding to off-target proteins may be a reason for non-specific side effects in normal tissues. For this reason, web-server based algorithms for drug target identification may optionally be used.

With this strategy, it may be estimated whether or not an identified drug candidate binds specifically to the corresponding target protein. The virtual drug screening procedure described herein may be mainly based on rigid docking approaches, i.e. conformational changes during binding of a drug to its target protein are preferably not considered. For this reason, also flexible docking techniques may be considered to be included in this screening program (e.g., molecular dynamics simulations). In selected cases, the results obtained by this virtual screening process may be experimentally verified. Using recombinant proteins, the binding of promising drug candidates may be investigated by appropriate techniques such as microscale thermopheresis, surface plasmon resonance spectroscopy, isothermal calorimetry etc.

In a preferred embodiment, step (v) of determining the binding affinity of each compound to the one or more docking spaces comprises:

(a) generating a 3D grid box of each docking space of the mutated protein and of each compound, wherein each grid box comprises grid points defined in all three dimensions that provide pieces of information selected from the group consisting of charges, partial charges, the ability to form hydrogen bonds, the ability to form pi-pi-electron interactions, and the ability to form van-der-Waals forces;

(b) fitting each 3D structure of a compound with the one or more docking spaces in a manner that the 3D structure of the compound can rotate and scans over each docking space;

(c) determining the binding energy between each compound and each docking space at each grid point and calculating binding affinity for each compound at each 3D orientation with each docking space; and

(d) determining the lowest binding affinity for each compound-protein interaction.

In a preferred embodiment, the method further comprises the following steps:

defining one or more docking spaces of the structure of the wild-type or homolog protein of step (ii) each corresponding to the respective docking spaces of the structure of the mutated protein of step (iii);

fitting the compounds with these one or more docking spaces;

determining the lowest binding energy of each compound to these one or more docking spaces and thereby determining the binding affinity;

comparing the binding affinity of each compound to the docking spaces of the mutated and of the wild-type or homolog compound; and

identifying one or more compounds having a higher binding affinity to the docking space of the wild-type or homolog protein than to the corresponding docking space of the mutated protein.

The one or more docking spaces of the mutated protein of interest may embrace the whole protein structure or a part thereof. In a preferred embodiment, a docking space embraces the whole protein, the surface of the whole protein optionally including one or more potential binding pockets or only the surrounding area of the pharmacophore binding site.

In a preferred embodiment, the method may comprise the following steps:

Isolation of RNA from diseased tissue, in particular neoplasm, cells or tissue derived from the patient

Determination of a mutational profile by RNA-sequencing

Examination whether three-dimensional protein crystal structures are available encoded by genes found to be mutated in from the Comparison of the mutational profile obtained from RNA sequencing with protein crystal structures of the correspondingly affected diseased tissue, in particular neoplasm, proteins.

Generation of mutation-specific protein homology models that resemble the mutated genes in individual diseased tissues, in particular neoplasms.

Bioinformatic screening of all FDA-approved drugs and other substances that preferentially bind with high affinity to these mutated proteins.

Inspection of scientific literature databases, whether the top-ranked drugs have been described to be cytotoxic towards cancer cells.

Decision making of the attending physician which drug can be chosen to treat individual diseased tissues, in particular neoplasms, with specific gene mutations.

In general, this technical procedure is applicable for all diseased, in particular neoplastic, entities (e.g., tumor entities such as, e.g., hematopoietic tumors, carcinoma, sarcoma, metastases, ascites, pleura effusions etc., as well as other diseases in which such repurposing may be useful in an adopted manner.

Then, one or more compounds specifically binding to the mutated protein may be identified (step (vi)). Optionally, one or more threshold levels may be set to distinguish the candidate compounds from the less-suitible compounds. It will be understood that such threshold levels are adapted to the individual purpose.

The person skilled in the art will select threshold, if usable, levels accordingly. Typically, the highest (selective) binding affinities to the mutated protein of interest indicate a good usability of a compound.

Optionally, the method may comprise one or more further steps for further assuring the medicinal usability of the one or more candidate compounds identified in step (vi). By using databases and computer algorithms, the identified drug candidate compounds may be, optionally, assessed for their toxicity profile and their potential interaction with other potentially co-medicated drugs.

In a preferred embodiment, the method further comprises the step (vii) of determining toxicological and pharmacologic properties of the compounds identified in step (vi) from one or more databases and identifying a compound of comparably low toxicity and, optionally, high pharmacologic activity in antineoplastic treatment.

This optional further step may be performed by any means. It may be the inspection of scientific literature databases, whether the top-ranked drugs have been described to be cytotoxic towards cancer cells.

In many cases, drugs approved for diseases other than diseased tissues, in particular neoplasms, have been described in the literature to exert also cytotoxic activity against neoplastic (e.g., tumor) cells. These published data may serve as confirmation that the compounds identified by the method of the present invention may indeed bear antineoplastic effects. Common database such as those selected from the group consisting of PubMed, Scopus, SciFinder and Google Scholar and other professional data mining tools and software will support the high-throughput screening of published literature, may be used for this evaluation step. Also this step may be conducted in a computer-assisted manner. It may, for instance be or comprise screening the internet or one or more locally stored databases for the searched pieces of information.

In a preferred embodiment, the compounds of the selection of compounds (of step (iv) of the method of the present invention) are approved for one or more pharmaceutical purposes. In a preferred embodiment, the compounds of the selection of compounds (of step (iv) of the method of the present invention) are approved for one or more pharmaceutical purposes other than antineoplastic activity and are not approved as antineoplastic agents. In other words, the compounds of the selection of compounds (of step (iv) of the method of the present invention) may preferably be pre-selected to have one or more of the aforementioned properties. In a preferred embodiment, the compounds are synthetical or semi-synthetical origin. Alternatively, the compounds may be of natural origin.

Finally a decision may be made which compound or combination of compounds to choose for a treatment. This step may be performed in a computer-assisted manner. For this purpose, the compounds identified in step (vi) may be further assessed for their described effects and/or undesired side-effects.

In a preferred embodiment, the information obtained may then serve as a support for decision-making by an automated devices like informational simulators of biological processes on the basis of the overall of clinical, laboratory and other information available on the individual patient as well as on criteria like availability, toxicity profile, side effects and drug interaction risk to serve as a generator of drug candidates for individual precision medicine in oncology and other fields. Also here threshold levels may be set. It will be understood that such threshold levels are adapted to the individual purpose. The person skilled in the art will select threshold, if usable, levels accordingly. Typically, the highest (selective) binding affinities to the mutated protein of interest in combination with low undesired side-effects (as described in one or more databases) and, optionally, indication of usability for antineoplastic treatment (as described in one or more databases) indicate a good usability of a compound.

The method of the present invention may also provide information of which dose of the one or more compounds should be used in the individual. Also here threshold levels may be set. It will be understood that such threshold levels are adapted to the individual purpose. The person skilled in the art will select threshold, if usable, levels accordingly. A balance between good pharmaceutical activity and rather low toxicity is desired. Also this step may be conducted in computer-assisted manner. As indicated above, preferably an antineoplastic agent or a combination of two or more thereof are provided. This, in a preferred embodiment, the method is a method for identifying an antineoplastic agent which has antineoplastic activity against the neoplasm, wherein said antineoplastic agent is or comprises one or more compounds identified in any of steps (vi) or (vii).

It will be understood that the present invention also provides novel and particularly beneficial antineoplastic agents or combinations of two or more thereof, in particular antineoplastic agents or combinations of two or more thereof for use in a method for treating a diseased tissue, in particular neoplasm, in an individual.

It will be understood that the present invention provides novel and particularly beneficial pharmaceutical compositions. This, in a preferred embodiment, the method of the present invention further comprises the step of preparing a pharmaceutical composition. This step may comprise combining a compound identified in any of steps (vi) or (vii) with a pharmaceutically acceptable carrier.

Accordingly, a further aspect relates to a pharmaceutical composition comprising one or more compounds identified in any of steps (vi) or (vii) of the method of the present invention and a pharmaceutically acceptable carrier.

As used herein, the term “pharmaceutically acceptable carrier” may refer to any substance that may support the pharmacological acceptance of the inhibitor. The pharmaceutical composition may be prepared for any type of administration such as, e.g., for oral administration, nasal administration, administration by means of injection (e.g., intravenous (i.v.), intraarterial (i.a.), intraperitoneal (i.p.), intramuscular (i.m.), subcutaneous (s.c.), intrathecal and/or intravitreal injection), subcutaneous administration, rectal administration and/or administration by means of inhalation. A pharmaceutical composition may be pharmaceutically formulated in a dry form (e.g., as a powder, a tablet, a pill, a capsule, a chewable capsule, etc.) or a liquid (e.g., a spray, a syrup, a juice, a gel, a liquid, a paste, an injection solution, an aerosol, an enema, etc.)

A pharmaceutically acceptable carrier may be a solvent with no or low toxicity such as, e.g., an aqueous buffer, saline, water, dimethyl sulfoxide (DMSO), ethanol, vegetable oil, paraffin oil or combinations thereof. Furthermore, the pharmaceutically acceptable carrier may contain one or more detergent(s), one or more foaming agent(s) (e.g., sodium lauryl sulfate (SLS), sodium doceyl sulfate (SDS)), one or more coloring agent(s) (e.g., TiO₂, food coloring), one or more vitamin(s), one or more salt(s) (e.g., sodium, potassium, calcium, zinc salts), one or more humectant(s) (e.g., sorbitol, glycerol, mannitol, propylenglycol, polydextrose), one or more enzyme(s), one or more preserving agent(s) (e.g., benzoic acid, methylparabene), one or more texturing agent(s) (e.g., carboxymethyl cellulose (CMC), polyethylene glycol (PEG), sorbitol), one or more emulsifier(s) , one or more bulking agent(s), one or more glacing agent(s), one or more separating agent(s), one or more antioxidant(s), one or more herbal and plant extract(s), one or more stabilizing agent(s), one or more polymer(s) (e.g., hydroxypropyl methacrylamide (HPMA), polyethylene imine (PEI), carboxymethyl cellulose (CMC), polyethylene glycol (PEG)), one or more uptake mediator(s) (e.g., polyethylene imine (PEI), dimethyl sulfoxide (DMSO), a cell-penetrating peptide (CPP), a protein transduction domain (PTD), an antimicrobial peptide, etc.) one or more antibody/antibodies, one or more sweetener(s) (e.g., sucrose, acesulfam K, saccharin Na, stevia), one or more counterstain dye(s) (e.g., fluorescein, fluorescein derivatives, Cy dyes, an Alexa Fluor dyes, S dyes, rhodamine, quantum dots, etc.), one or more gustatory substance(s) and/or one or more fragrance(s).

As indicated above, the compound identified in any of steps (vi) or (vii) of the method of the present invention, an antineoplastic agent of the present invention and the pharmaceutical composition of the present invention may be particularly well used for treating a diseased tissue, in particular neoplasm, in an individual.

A further aspect relates to a compound identified in any of steps (vi) or (vii) of the method of the present invention, an antineoplastic agent of the present invention or a pharmaceutical composition of the present invention for use in a method for treating a diseased tissue, in particular neoplasm, in an individual.

In other words, the present invention relates to a compound identified in any of steps (vi) or (vii) of the method of the present invention, an antineoplastic agent of the present invention or a pharmaceutical composition of the present invention for use in a method for treating an individual suffering from a diseased tissue, in particular neoplasm, in particular cancer.

In still other words, the present invention relates to a method for treating an individual suffering from a diseased tissue, in particular neoplasm, in particular cancer, said method comprising the administration of a compound identified in any of steps (vi) or (vii) of the method of the present invention, an antineoplastic agent of the present invention or a pharmaceutical composition of the present invention in a pharmaceutically effective amount.

As used herein, the terms “antineoplastic agent”, “anticancer agent”, “antineoplastic drug”, “anticancer drug”, “anticancer compound”, “antineoplastic compound” and equivalents thereof may be understood interchangeably in the broadest sense as any agent that is suitible for treating a malignant tumor (i.e., cancer). Exemplarily, such antineoplastic agent may be selected from the group consisting of chemotherapeutics, hormones and analogue thereof and other antineoplastic agent. Exemplarily, such antineoplastic agent may be selected from the group consisting

of platins (e.g., cisplatin, carboplatin, oxaliplatin), anti-metabolites (e.g., azathioprine, 6-mercaptopurine, mercaptopurine, 5-fluorouracil, pyrimidines, thioguanine, fludarabine, floxuridine, cytosine arabinoside (cytarabine), pemetrexed, raltitrexed, pralatrexate, methotrexate), further alkylating agents (e.g., chlorambucil, Ifosfamide mechlorethamine, cyclophosphamide), statins (e.g., cerivastatin, simvastatin, lovastatin, somatostatin, fluvastatin, nystatin, rosuvastatin, atorvastatin, pravastatin, pitavastatin, pentostatin,),terpenoids and plant alkaloids (e.g., vinca alkaloids (vincristine, vinblastine, vinorelbine, vindesine), taxanes (e.g., paclitaxel), cytoxan), topoisomerase inhibitors (e.g., camptothecins: irinotecan, topotecan, etoposide, etoposide phosphate, teniposide), melphalan, other antineoplastica (e.g., doxorubicin (adriamycin), doxorubicin lipo, epirubicin, bleomycin)), actinomycin D, aminoglutethimide, amsacrine, anastrozole, antagonists of purine and pyrimidine bases, anthracyclines, aromatase inhibitors, asparaginase, antiestrogens, bexarotene, buserelin, busulfan, camptothecin derivatives, capecitabine, carmustine, cladribine, cytarabine, cytosine arabinoside, alkylating cytostatics, dacarbazine, daunorubicin, docetaxel, epirubicin, estramustine, etoposide, exemestane, fludarabine, fluorouracil, folic acid antagonists, formestane, gemcitabine, glucocorticoids, goserelin, hormones and hormone antagonists, hycamtin, hydroxyurea, idarubicin, irinotecan, letrozole, leuprorelin, lomustine, mercaptopurine, miltefosine, mitomycins, mitosis inhibitors, mitoxantrone, nimustine, procarbazine, tamoxifen, temozolomide, teniposide, testolactone, thiotepa, topoisomerase inhibitors, treosulfan, tretinoin, triptorelin, trofosfamide, cytostatically active antibiotics, everolimus, pimecrolimus, tacrolimus, azithromycin, spiramycin, sirolimus (rapamycin), roxithromycin, ascomycin, bafilomycin, erythromycin, midecamycin, josamycin, concancamycin, clarithromycin, troleandomycin, folimycin, tobramycin, mutamycin, dactinomycin, dactinomycin, rebeccamycin, 4-hydroxyoxycyclophosphamide, bendamustine, thymosin α-1, aclarubicin, fludarabine-5′-dihydrogen phosphate, hydroxycarbamide, aldesleukin, pegaspargase, cepharanthine, epothilone A and B, azathioprine, mycophenolate mofetil, c-myc antisense, b-myc antisense, betulinic acid, camptothecin, melanocyte stimulating hormone (α-MSH), activated protein C, IL-1β inhibitor, fumaric acid and esters thereof, dermicidin, calcipotriol, taclacitol, lapachol, β-lapachone, podophyllotoxin, betulin, podophyllic acid 2-ethyl hydrazide, sagramostim, (rhuGM-CSF), peginterferon α-2b, lenograstim (r-HuG-CSF), filgrastim, macrogol, cephalomannine, selectin (cytokine antagonist), CETP inhibitor, cadherins, cytokinin inhibitors, agrostistachin, 17-hydroxyagrostistachin, ovatodiolids, 4,7-oxycycloanisomelic acid, baccharinoids B1, B2, B3 and B7, tubeimoside, bruceanol A, B and C, bruceantinoside C, yadanziosides N and P, isodeoxyelephantopin, tomenphantopin A and B, coronarin A, B, C and D, ursolic acid, COX inhibitor (e.g., COX-2 and/or COX-3 inhibitor), angiopeptin, ciprofloxacin, fluroblastin, bFGF antagonists, probucol, prostaglandins, 1,11-dimethoxyeanthin-6-one, 1-hydroxy-11-methoxycanthin-6-one, scopoletin, colchicine, NO donors, pentaerythrityl tetranitrate, sydnonimines, S-nitroso derivatives, staurosporine, β-estradiol, α-estradiol, estriol, estrone, ethinyl estradiol, fosfestrol, medroxyprogesterone, estradiol cypionates, cudraisoflavone A, curcum in, dihydronitidine, nitidine chloride, 12-beta-hydroxypregnadiene-3,20-dione bilobol, ginkgol, ginkgolic acid, helenalin, indicine, indicine-N-oxide, lasiocarpine, inotodiol, glycoside 1a, justicidin A and B, larreatin, malloterin, mallotochromanol, isobutyrylmallotochromanol, marchantin A, maytansine, lycoridicin, margetine, pancratistatin, liriodenine, bisparthenolidine, oxoushinsunine, aristolactam-All, estradiot benzoates, tranilast, kamebakaurin, verapamil, ciclosporin A, paclitaxel and derivatives thereof such as 6-α-hydroxy paclitaxel, baccatin, taxotere, mofebutazone, acemetacin, diclofenac, lonazolac, dapsone, o-carbamoyl-phenoxy-acetic acid, lidocaine, ketoprofen, mefenamic acid, piroxicam, meloxicam, chloroquine phosphate, penicillamine, hydroxychloroquine, auranofin, sodium aurothiomalate, oxaceprol, celecoxib, β-sitosterol, ademetionine, myrtecaine, polidocanol, nonivamide, levomenthol, benzocaine, aescin, elipticine, D-24851 (Calbiochem), colcemid, cytochalasin A-E, indanocine, nocodazole, bacitracin, vitronectin receptor antagonists, azelastine, free nucleic acids, nucleic acids incorporated into virus transmitters, DNA and RNA fragments, plasminogen activator inhibitor-1, plasminogen activator inhibitor-2, antisense oligonucleotide, VEGF inhibitors, IGF-1, active agents from the group of antibiotics such as cefadroxil, cefazolin, cefaclor, cefoxitin, gentamicin, penicillins, dicloxacillin, oxacillin, sulfonamides, metronidazole, antithrombotics, argatroban, aspirin, abciximab, synthetic antithrombin, bivalirudin, coumadin, enoxaparin, GpIIb/IIIa platelet membrane receptor, antibodies to factor Xa inhibitor, heparin, hirudin, r-hirudin, PPACK, protamine, prourokinase, streptokinase, warfarin, urokinase, vasodilators, dipyramidole, trapidil, nitroprussides, PDGF antagonists, triazolopyrimidine, seramin, ACE inhibitors, captopril, cilazapril, lisinopril, enalapril, losartan, thioprotease inhibitors, prostacyclin, vapiprost, interferon α, β and γ, histamine antagonists, serotonin blockers, apoptosis inhibitors, apoptosis regulators, NF-kB, Bcl-xL antisense oligonucleotides, halofuginone, nifedipine, tocopherol, molsidomine, tea polyphenols, epicatechin gallate, epigallocatechin gallate, boswellic acids and derivatives thereof, leflunomide, anakinra, etanercept, sulfasalazine, tetracycline, triamcinolone, procainimide, retinoic acid, quinidine, disopyramide, flecainide, propafenone, sotalol, amiodarone, natural and synthetically obtained steroids such as withaferin A, bryophyllin A, inotodiol, maquiroside A, mansonine, strebloside, hydrocortisone, betamethasone, dexamethasone, fenoprofen, ibuprofen, indomethacin, naproxen, phenylbutazone, acyclovir, ganciclovir, zidovudine, antimycotics, clotrimazole, flucytosine, griseofulvin, ketoconazole, miconazole, terbinafine, chloroquine, mefloquine, quinine, natural terpenoids, hippocaesculin, barringtogenol-C₂₁-angelate 14-dehydroagrostistachin, agroskerin, hyptatic acid A, zeorin, strychnophylline, usambarine, usambarensine, daphnoretin, lariciresinol, methoxylariciresinol, syringaresinol, umbelliferone, afromoson, acetylvismione B, desacetylvismione A, vismione A and B, iso-iridogermanal, maytenfoliol, effusantin A, excisanin A and B, longikaurin B, sculponeatin C, kamebaunin, leukamenin A and B, 13,18-dehydro-6-alpha-senecioyloxychaparrine, taxamairin A and B, regenilol, triptolide, cymarin, apocymarin, aristolochic acid, anopterin, hydroxyanopterin, anemonin, protoanemonin, berberine, cheliburin chloride, cicutoxin, sinococuline, combrestatin A and B, periplocoside A, ghalakinoside, deoxypsorospermin, psychorubin, ricin A, sanguinarine, manwu wheat acid, methylsorbifolin, chromones of spathelia, stizophyllin, akagerine, dihydrousambaraensine, hydroxyusambarine, strychnopentamine, a pharmaceutically acceptable salt of any thereof, and a combination of two or more thereof or two or more pharmaceutically acceptable salts thereof.

An antineoplastic agent may also be an agent suitible for immunotherapy of malignant tumors. An agent suitible for immunotherapy of malignant tumors may be understood in the broadest sense as any agent suitible to stimulate the immune system to treat malignant tumors. It may be active, passive or a mixture of both (hybrid). In this context, immunotherapy may base on the detectability of diseased tissue-associated, in particular neoplasm-associated, antigens (often also designated as tumour-associated antigens (TAAs)). Active immunotherapy may direct the immune system to attack diseased, in particular neoplastic, cells by targeting diseased tissue-associated, in particular neoplasm-associated, antigens. Passive immunotherapies may enhance existing antineoplastic responses and include the use of antibodies or fragments or variants thereof, immune cells (e.g., lymphocytes (e.g., T-lymphocytes, B-lymphocytes), natural killer cells, lymphokine-activated killer cells, cytokine-activated killer cells, cytotoxic T cells and dendritic cells) and/or cytokines, in particular (optionally humanized) monoclonal antibodies or fragments thereof. Depending on the individual setup, such antibodies or fragments or variants thereof, immune cells and/or cytokines may lead to antibody-dependent cell-mediated cytotoxicity, may activate the complement system, and/or may prevent a receptor from interacting with its ligand. Thereby, in some setups, the targeted cell may be triggered into apoptosis. Examples for antibodies usable in the context of immune therapy include alemtuzumab, ipilimumab, nivolumab, ofatumumab and rituximab. Antibodies or fragments or variants thereof may optionally also be conjugated (e.g., by a radioactive ion). Additionally or alternatively, also dendritic cell therapy may be used. Additionally or alternatively, also cytokines, keyhole limpet hemocyanin, Freund's adjuvant, Bacillus Calmette-Guérin (BCG) vaccine and/or peginterferon alfa-2a may be used. Alternatively or additionally, also an antineoplastic vaccine may be used such as, e.g., a vaccine made of diseased, in particular neoplastic, tissue or an artificial vaccine (e.g., polypeptide-based, polynucleotide-based, glycoside-based, etc.). The person skilled in the art will be aware of several further agents suitible for immunotherapy of malignant tumors usable in the context of the present invention.

As described above, at least some steps of the method of the present invention are preferably conducted in a computer-assisted manner. It will be understood that a special combination or algorithms is required for this purpose. Thus, also the combination or algorithms bar special technical effects.

Therefore, a further aspect relates to a computer program comprising instructions which, when the program is executed by a computer, cause the computer to carry out at least steps (iv) and (v) of the method of the present invention.

The computer program may be stored on any storage device such as, e.g., a computer hard disc, the working memory, a USB stick, a CD ROM etc. Thus, the present invention also refers to a storage device comprising, stored thereon, a computer program comprising instructions which (when the program is executed by a computer) cause a computer to carry out at least steps (iv) and (v) of the method of the present invention.

In a preferred embodiment, the computer program comprises instructions which, when the program is executed by a computer, cause the computer to carry out at least steps (iii)-(v), at least steps (iii)-(vi) or at least steps (iv)-(vi), at least steps (ii)-(v), at least steps (ii)-(vi) or at least steps(ii) and (iv)-(vi) of the method of the present invention

The following Examples and claims are intended to provide illustrative embodiments of the present invention described and claimed herein.

### EXAMPLES

Materials and Methods

1 Isolation of RNA from Tumor Cells or Tissue Derived from the Patient.

The test sample was taken up directly after removal into RNA stabilization solution. Total RNA isolation was performed with a column-based extraction procedure to obtain pure RNA without DNA digestion. The quality of the RNA was proven and the threshold set was an RNA integrity score of 6.8 or higher. To exclude ribosomal RNA sequences from further analysis, the RNA was hybridized with eukaryotic ribosomal RNA biotin-labeled oligonucleotide probes to deplete ribosomal RNA from total RNA. For the preparation of poly A+ RNA, streptavidin-coated magnetic beads coupled with oligo-dT were used. Five micrograms total RNA were mixed with beads and RNA purification beads and incubated. After incubation for 5-10 min, the beads were pelleted on a magnetic stand and the supernatant can be discarded. After washing the beads with washing buffer, the beads were resuspended in elution buffer to elute RNA from the beads. Then, a binding process with binding buffer took place again. The RNA bead mix was eluted again and the RNA was fragmented by heat treatment at 65° C. for 5-10 min. the elution and prime mix contains hexamers with random sequences and reverse transcriptase and was used to start cDNA synthesis from the RNA templates the supernatant was transferred to the master mix and put into a PCR plate with the barcode sequence. If the thermal cycling was finished, the RNA strand was removed and substituted by a second cDNA strand. Using specific beads, double-stranded cDNA was separated from RNA and the reaction mix. Overhanging strand ends from fragmentation was finally digested by 3′-5′ exonuclease to blunt ends. 5′ overhangs were filled to blunt ends by polymerase.

This method was applicable for cells, solid tissues, blood and other body fluids. Total RNA quality and quantity was evaluated by a microfluidics-based platform. After loading, the sample migrates through micro-channels to electrophoretically separate the sample components. The fluorescent probe intercalates into RNA strands and the fluorescence was recorded. The example does not only refer to coding mRNA, but also for non-coding RNA and epigenetically changed DNA sequences, as well as proteins, peptides, lipids, and all other metabolic chemical substances.

2. Determination of a Mutational Profile and Transcript Abundance by RNA-Sequencing

End-repaired, A-tailed and adaptor-ligated cDNA was PCR-amplified by 10 cycles. The library was sequenced in paired-end mode (2×100 bp) using commercial RNA sequencing systems. The resulting sequences were aligned to a reference genome. Discrepencies concerning point mutations, deletions amplifications insertions etc. were recorded. Normalized RNA expressions were quantified using the RPKM measure. RPKM values for transcripts and the ratios of transcripts were taken into consideration to calculate the overall RPKM value for each gene.

3. Examination whether Three-Dimensional Protein Crystal Structures are Available

Several proteins encoded by genes found to be mutated in from the comparison of the mutational profile obtained from RNA sequencing with protein crystal structures of the correspondingly affected proteins. In cases, where isoforms or splicing variants of target proteins are available, several homology models were prepared in parallel. Information on alterations in helices, disulfide bridges secondary loop structures, distortions of beta-sheets etc. may change the protein conformation and therefore may alter the binding properties of drugs. In selected cases, sequence alignments of proteins from different species were performed, because the interspecies comparison can give information of interest about commonly conserved and unique sequence motifs, key amino acid positions in the pharmacophore domains, identical location of helix bending residues etc. Furthermore, co-crystallization of target proteins with other binding proteins, small molecules, antibodies, peptides etc. was also considered, since they may not only stabilize the protein of interest, but also change its conformation from an inactive in to an active state and vice versa.

Furthermore, electrostatic potential maps were calculated to determine hot spots of electron density that may interfere with binding properties of affected amino acid residues. This information may be of interest to find the most appropriate small molecule inhibitor drugs.

4. Generation of Mutation-Specific Protein Homology Models that Resemble the Mutated Genes in Individual Tumors.

The method described herein was conducted on a high performance computer running on Linux etc. to meet the requirements of the multi-stage process of protein modeling. For several calculations, the supercomputer MOGON II (Mainz, Germany) was used. Either the crystallography-based structures of human target proteins or corresponding crystal structures from other species were used for homology modeling. Internet-based databases for protein crystal structures were searched for the availability of three-dimensional structures that could be used as templates to create models of patient-specific mutations. In cases, where crystal structures of human proteins were not available, corresponding protein structures from other species may serve a template to generate human protein homology models. Homology modeling was based on the creation of three-dimensional models of proteins with known amino acid sequence, but unknown crystal structure. A precondition for homology modeling was the existence of a crystal structure of a related protein. With an available crystal structure (e.g. a wild-type protein), the sequence of the known (wild-type) protein can be aligned to the protein with the still unknown 3D-structure (e.g. the mutant counterpart of the wild-type protein).

Based on the known crystal structure of the wild-type protein, a hypothetical 3D-structure of the corresponding mutant protein can be calculated. This homology model was as better as more conserved are the amino acid sequences of known and unknown proteins. As a first step, the protein sequence was downloaded from a corresponding website, (e.g. UniProt) in FASTA format. Then, the known 3D structure of the related protein, which should serve as template, was downloaded and both protein sequences were compared using BLAST (Basic Local Alignment Search Tool) and ClustalW2. Crystal structures or homology models of wild-type proteins were then modified by insertion of the amino acid exchanges delineated from RNA-sequencing of the specific patient tumor. The subsequent homology models of mutated proteins were created using the alignment file with appropriate alignment programs.

The Swiss-MODEL structure assessment tool was then used to select the best homology model for molecular docking. Model evaluation was done with the help several tools (Anolea, GROMOS, QMEAN, DFIRE etc.). In a cellular environment. proteins existed in a hydrated form. Therefore, hydrogens were added to Asn and Gln residues.

5. Bioinformatic Screening of a Library of FDA-Approved Drugs that Preferentially Bind with High Affinity to these Mutated Proteins.

A high performance Linux-based computer cluster was desirable for running virtual drug screening campaigns in sufficiently short time to deliver results to the decision-making physicians A library of FDA-approved drugs (>1500 compounds) was used to investigate the binding of drug to the mutation-specific protein homology models by means of specific virtual drug screening programs. These FDA-approved drugs do not only contain anticancer drugs, but drugs that were used for all kinds of diseases. The idea was that drugs frequently do not act in a mono-specific manner, but have broader activity spectra. Therefore, drugs for a specific disease indication may also inhibit related mutated proteins as in cancer. These inhibitory drugs were identified by bioinformatic calculation of drug-protein binding affinities. With this approach, approved drugs could be used off-label to treat individual tumors according to their individual mutations. This was the main concept of the present drug repurposing invention. Several algorithms to identify the best binding drugs with independent techniques were used. As an example, the 10 top-ranked out of >1500 FDA-approved drugs with highest affinities was selected. Homology-modeled mutant patient-specific proteins were set as rigid receptor molecules.

The prepared output files indicated information on atomic partial changes, torsion degrees of freedom and different atom types was added, e.g. aliphatic and aromatic carbon or polar atoms forming hydrogen bonds such as in PDQT format. In cases, where target proteins contain known pharmacophore sites, grids around selected amino acid residues of that pharmacophore were defined to calculate drug binding (defined docking approach). In those cases, where no drug-binding site of a target protein was known, interaction energies for the whole protein (blind docking approach) were first calculated. The region showing with the highest binding affinity were then be used to set a grid and a defined docking followed as a second step. The grid box was constructed to define docking spaces.

The dimensions of the grid box was set around the entire protein (blind docking approach) or around defined pharmacophore sites (defined docking approach) in a manner that the ligand could freely move and rotate in the docking space. The grid box consists of for instance 126 grid points in all three dimensions (X, Y and Z axes) separated by a distance of for instance 1 between each one. Energies at each grid point were then evaluated for each atom type present in the ligand, and the values were used to predict the energy of a particular ligand configuration. Three independent docking calculations were conducted, with 25,000,000 energy evaluations and 250 runs by using the Lamarckian Genetic Algorithm. The corresponding binding energies and the number of conformations in each cluster were attained from the docking log files (dig). The corresponding lowest binding energies (LBE) were obtained from the docking log files (dig), and mean values ±SD were calculated.

The docking results were visualized to prove the correct binding of the drugs to the relevant drug-binding sites of the mutated tumor proteins. By using databases and computer algorithms, the identified drug candidates were examined for their toxicity profile and their potential interaction with other potentially co-medicated drugs. To prove the specificity of the identified candidate drugs for a given mutated target protein, the binding of this drug to both the mutated and the wild-type protein models was performed. If more models are available (splice variants, proteins from other species), they were also be included in the docking procedure to obtain the best possible information about binding of this drug to the target protein.

To set up molecular docking, the data were first copied into the corresponding folder of the ligand docking program. Before doing so, two-dimensional chemical structures were converted to three-dimensional ones using appropriate software programs. The energy of the compound was minimized and the new structure saved as mol file. For subsequent molecular docking, the files of the ligands were prepared in pdbqt format, the ones of the target proteins in gpf, glg, and dpf file format. Then, the script for running the docking was prepared. Each calculation has a maximum runtime of five days (=7200 min). Each calculation was started using the script. The results of the running jobs were saved in the directory of the ligand. After finalizing the jobs, the results can be copied to personal computers. For docking campaigns of more than 64 ligands, a node-long script was used. Furthermore, it was taken into account that a drug that has been identified to bind to a given target protein found in the tumor genome of a patient might not only bind to this protein but also to several others. Binding to off-target proteins may be a reason for non-specific side effects in normal tissues. For this reason, web-server based algorithms for drug target identification were used. With this strategy, it could be estimated whether or not an identified drug candidate binds specifically to the corresponding target protein. The virtual drug screening procedure described herein was mainly based on rigid docking approaches, i.e. conformational changes during binding of a drug to its target protein were not considered. For this reason, flexible docking techniques were also considered to be included in this screening program (e.g. Molecular Dynamics simulations). In selected cases, the results obtained by this virtual screening process were experimentally verified. Using recombinant proteins, the binding of promising drug candidates was investigated by appropriate techniques such as microscale thermopheresis, surface plasmon resonance spectroscopy, isothermal calorimetry etc.

6. Inspection of Scientific Literature Databases, whether the Top-Ranked Drugs have been Described to be Cytotoxic Towards Cancer Cells.

In many cases, drugs approved for diseases other than cancer have been described in the literature to exert also cytotoxic activity against tumor cells. These published data may serve as confirmation that the drugs identified by the present technical procedure may indeed be able to kill cancer cells. Common databases were screened such as PubMed, Scopus, SciFinder, Google Scholar etc. and other professional data mining tools and software supported the high-throughput screening of published literature.

7. Decision Making of the Attending Physician which Drug can be Chosen to Treat Individual Tumors with Specific Gene Mutations.

The information obtained then served as a support for decision-making by physicians, tumor boards, other decision makers or automated devices like informational simulators of biological processes on the basis of the overall of clinical, laboratory and other information available on the individual patient as well as on criteria like availability, toxicity profile, side effects and drug interaction risk to serve as a generator of drug candidates for individual precision medicine in oncology and other fields.

Results

Biopsy material of a liver metastasis of a breast carcinoma has been obtained from a 50-year old patient. The patient had received various chemotherapies over more than a decade and showed extended metastases. The tumor was progressive and not responsive to the current chemotherapies anymore while tumor marker Ca15.3 rose to 22,230 units/ml. PDL Antibody therapy (Keytruda, 100 mg) did not change tumor markers in controls. The responsible tumor board recommended NAP-Paclitaxel with little hope that this would substantially change the course of the disease.

To gain further options of therapy, biopsy of liver metastasis was performed and test results were obtained as shown below. Irinotecan was identified as a candidate for treatment according to the test results and infused in two-weeks intervals according to the standard protocols. After that, CA15.3 went down to 1513 units and malignant ascites was markedly reduced. After half a year, the patient showed stable disease and clinical well-being. She took a two week vacation to France and feels well.

The complete transcriptome with >20,000 mRNA species was sequenced. RNA sequencing of the presented patient showed a total number of 47,562 mutations.

A database of 2483 proteins that are described in the literature as being cancer-related was prepared.

611 RNA mutations in the presented patient led to amino acid changes in cancer-related proteins of this database.

From these 2483 proteins, 85 DNA repair proteins were excluded, because mutated DNA repair function cannot be pharmacologically regained.

From the remaining 2398 proteins, the 561 amino acid mutations were distributed among the proteins as follows:

253 proteins with 1 amino acid mutation;

69 proteins with 2 amino acid mutations;

18 proteins with 3 amino acid mutations;

19 proteins with 4 or more amino acid mutations.

Of the affected 359 proteins, 12 three-dimensional crystal structures were available. As more and more crystal structures of the human proteome were determined, the number of testable proteins increased over time. This means that the power of identification of effective repurposing drugs increased with increasing knowledge about the availability of three-dimensional protein structures.

The wild-type sequences of these 12 proteins were used, included the mutations and prepared three-dimensional homology models of these mutated proteins. Ten of the mutated proteins carries each one amino acid change. Two further proteins carried two amino acid mutations:

| Mutated genes | Type of mutation | Amino acid exchange |
|---|---|---|
| ABCA1 | missense variant | V825I |
| ABCB1 | missense variant | S893A |
| AKR1C3 | missense variant | E77G |
| ANKRD27 | missense variant | P761G |
| ANXA5 | missense variant | I81T |
| APOBEC3B | missense variant | K146T |
| ATP7B | missense variant | S406A und V1140A |
| CASP8 | missense variant | K344H |
| HLA-B | missense variant | H140S und I218V |
| NQO1 | missense variant | P187S |
| PARP1 | missense variant | V762A |
| TLR1 | missense variant | N248S |

All 12 homology models were subjected to virtual drug screening with >1500 FDA-approved drugs. This screening campaign resulted in 12 drug ranking lists. Each the top 10 drugs of all 12 drug ranking lists were inspected and searched for those drugs which appeared in more than one of these lists:

| How often | | |
|---|---|---|
| appearing in the | | |
| 12 top 10 lists | | Remarks |
| 9 | Conivaptan | Against low sodium levels |
| 7 | Venetoclax | antineoplastic agent; BCL-2 inhibitor |
| 6 | Nilotinib | tyrosine kinase inhibitor against |
| | | chronic myeloid leukaemia |
| 4 | Vumon | DNA topoisomerase type II inhibitor |
| 4 | Ponatinib | tyrosine kinase inhibitor against |
| | | chronic myeloid leukaemia and acute |
| | | lymphoblastic leukemia |
| 4 | Eltrombopag | against thrombocytopenia with chronic |
| | | immune (idiopathic) thrombocytopenic |
| | | purpura |
| 3 | Irinotecan | DNA topoisomerase type 1 inhibitor |
| 2 | Olaparib | PARP inhibitor, against breast cancer |
| 2 | Sirolimus | mTOR inhibitor |

As all of these drugs bind with high affinity to more than one mutated protein the identified drugs have a multi-specific target specificity. It can be expected that they were more active than mono-specific drugs that bind only to one single target. This approach was also applied by us for other conditions (cancers with one specific driver mutation, mutation-mediated inherited and somatic genetic diseases).

REFERENCES

Aronson J K. Old drugs—new uses. Br. J Clin. Pharmacol. 2007;64:563-565.

Ashburn T T, Thor K B. Drug repositioning: identifying and developing new uses for existing drugs. Nat. Rev. Drug Discov. 2004;3:673-683.

Dakshanamurthy S, Issa N T, Assefnia S, Seshasayee A, Peters O J, Madhavan S, Uren A, Brown M L, Byers S W. Predicting new indications for approved drugs using a proteochemometric method. J Med Chem. 2012;55(15):6832-6848.

Efferth T, Saeed M E M, Mirghani E, Alim A, Yassin Z, Saeed E, Khalid H E, Daak S. Integration of phytochemicals and phytotherapy into cancer precision medicine. Oncotarget 2017;8:50284-50304.

Hientz K, Mohr A, Bhakta-Guha D, Efferth T. The role of p53 in cancer drug resistance and targeted chemotherapy. Oncotarget 2017;8:8921-8946.

Kadioglu O, Saeed M, Kuete V, Greten H J, Efferth T. Oridonin Targets Multiple Drug-Resistant Tumor Cells as Determined by in Silico and in Vitro Analyses. Frontier in Pharmacology 2018;9:355. doi: 10.3389/fphar.2018.00355.

Kola I, Landis J. Can the pharmaceutical industry reduce attrition rates? Nat. Rev. Drug Discov. 2004;3:711-715.

Marin J J G, Lozano E, Herraez E, Asensio M, Di Giacomo S, Romero MR, Briz O, Serrano M A, Efferth T, Macias R I R. Chemoresistance and chemosensitization in cholangiocarcinoma. Biochim. Biophys. Acta 2018;1864:1444-1453.

Mbaveng A T, Kuete V, Efferth T. Potential of Central, Eastern and Western Africa Medicinal Plants for Cancer Therapy: Spotlight on Resistant Cells and Molecular Targets. Front. Pharmacol. 2017;8:343

Moehler T. Clinical experience with thalidomide and lenalidomide in multiple myeloma. Curr. Cancer Drug Targets 2012;12:372-390.

Mullard A. FDA drug approvals. Nat. Rev. Drug Discov. 2012; 11:91-94.

Schmidt F, Efferth T. Tumor Heterogeneity, Single-Cell Sequencing, and Drug Resistance. Pharmaceuticals (Basel) 2016;9.pii: E33.

Vargesson N. Thalidomide-induced teratogenesis: history and mechanisms. Birth Def. Res. Part C, Embryo Today: Rev. 2015;105:140-156.

Volm M, Efferth T. Prediction of Cancer Drug Resistance and Implications for Personalized Medicine. Front. Oncol. 2015;5:282.

Walther Z, Sklar J. Molecular tumor profiling for prediction of response to anticancer therapies. Cancer J. 2011;17:71-79.

WO 2003/057173.

WO 2001/035316.
The present listing of claims replaces all prior listings or versions of claims in the present application.

1. A method for identifying one or more compounds specifically binding to a target structure of a given diseased tissue, comprising:
(i) identifying a mutated gene in the transcriptome of said diseased tissue and identifying at least one mutation comprised in said mutated gene;
(ii) providing a three-dimensional (3D) structure of a wild-type or homolog protein expressed by a wild-type or homolog gene corresponding to the mutated gene identified in step (i);
(iii) determining a 3D structure of a mutated protein which is the expression product of the mutated gene identified in step (i) or one or more docking spaces thereof, comprising:
(a) adapting the amino acid sequence of the 3D structure of the wild-type or homolog protein of step (ii) to the expression product of the mutated gene identified in step (i) and defining one or more docking spaces of the obtained 3D structure of mutated protein, or
defining one or more docking spaces of the 3D structure of the wild-type or homolog protein of step (ii) and adapting the amino acid sequence of said one or more docking spaces to the expression product of the mutated gene identified in step (i);
(iv) providing 3D structures of a selection of compounds and fitting each 3D structure of each compound with the one or more docking spaces of step (iii);
(v) determining the binding affinity of each compound to the one or more docking spaces; and
(vi) identifying one or more compounds specifically binding to the mutated protein.

2. The method of claim 1, wherein the step (i) of identifying a mutated gene and the at least one mutation comprises:
(a) providing a sample from the diseased tissue containing mRNA;
(b) optionally isolating and/or purifying the mRNA;
(c) optionally generating cDNA from the mRNA by a polymerase chain reaction; and
(d) identifying at least one mutation by means of at least one step selected from the group consisting of:
sequencing the mRNA and/or the cDNA;
hybridizing the mRNA and/or the cDNA with a chip containing a variety of single-stranded nucleotides embracing mutated and non-mutated sequences; and
conducting a polymerase chain reaction with a number of primers including those specific for a particular mutation.

3. The method of claim 1, wherein the diseased tissue is a neoplasm.

4. The method of claim 1, wherein the mutated gene, the mutated protein, or a combination thereof is associated with the onset or progression of a neoplasm.

5. The method of claim 1, wherein the selection of compounds used in step (iv) comprises at least five compounds.

6. The method of claim 1, wherein:
the 3D structure of the wild-type or homolog protein of step (ii) is a crystal structure, a 3D NMR structure or a calculated hypothetical three-dimensional structure and is, optionally, obtained from a structure database; and/or
the mutation is a point mutation and the mutated protein differs from the non-mutated protein by a single amino acid moiety only and each docking space embraces the different single amino acid moiety.

7. The method of claim 1, wherein at least steps (ii)-(v) are conducted in a computer-assisted manner.

8. The method of claim 1, wherein at least one of the compounds of which 3D structures are provided in step (iv) is characterized by one or more of the properties selected from the group consisting of:
the compound has a molecular weight of not more than 1000 Da,
the compound is not approved as an antineoplastic agent,
the compound is has known pharmacokinetic properties, and
the compound is approved for one or more pharmaceutical purposes other than antineoplastic activity.

9. The method of claim 1, wherein step (v) of determining the binding affinity of each compound to the one or more docking spaces comprises:
(a) generating a 3D grid box of each docking space of the mutated protein and of each compound, wherein each grid box comprises grid points defined in all three dimensions that provide pieces of information selected from the group consisting of charges, partial charges, the ability to form hydrogen bonds, the ability to form pi-pi-electron interactions, and the ability to form van-der-Waals forces;
(b) fitting each 3D structure of a compound with the one or more docking spaces in a manner that the 3D structure of the compound can rotate and scans over each docking space;
(c) determining the binding energy between each compound and each docking space at each grid point and calculating binding affinity for each compound at each 3D orientation with each docking space; and
(d) determining the lowest binding affinity for each compound-protein interaction.

10. The method of claim 1, wherein the method further comprises the following steps:
defining one or more docking spaces of the structure of the wild-type or homolog protein of step (ii) each corresponding to the respective docking spaces of the structure of the mutated protein of step (iii);
fitting the compounds with these one or more docking spaces;
determining the lowest binding energy of each compound to these one or more docking spaces and thereby determining the binding affinity;
comparing the binding affinity of each compound to the docking spaces of the mutated and of the wild-type or homolog compound; and
identifying one or more compounds having a higher binding affinity to the docking space of the wild-type or homolog protein than to the corresponding docking space of the mutated protein.

11. The method of claim 1, wherein determining the binding affinity of each compound to the one or more docking spaces includes using Lamarckian Genetic Algorithm.

12. The method of claim 1, wherein a docking space embraces the whole protein, the surface of the whole protein optionally including one or more potential binding pockets or only the surrounding area of the pharmacophore binding site.

13. The method of claim 1, wherein the diseased tissue is compared with comparable healthy tissue.

14. The method of claim 13, wherein the comparable healthy tissue is obtained from the same individual as the diseased tissue.

15. The method of claim 13, wherein the comparable healthy tissue is obtained from another individual of the same species.

16. The method of claim 1, wherein the diseased tissue bears one or more genetic variations selected from the group consisting of one or more mutations, one or more different alleles, one or more polymorphisms, or combinations of two or more thereof, in comparison to corresponding healthy tissue.

17. The method of claim 1, wherein the diseased tissue bears one or more mutations associated with the disease state of the diseased tissue in comparison to corresponding healthy tissue.

18. The method of claim 13, wherein the comparison between the diseased tissue with comparable healthy tissue is comparing the specific binding of the one or more compounds to one or more target structures of a given diseased tissue with the binding of said one or more compounds to target structures which are the counterparts in healthy tissue of the one or more target structures of the given diseased tissue.

19. The method of claim 1, wherein said method further comprises the step (vii) of determining toxicological and pharmacologic properties of the compounds identified in step (vi) from one or more databases and identifying a compound of comparably low toxicity and, optionally, high pharmacologic activity in antineoplastic treatment.

20. The method of claim 19, wherein said method is a method for identifying an antineoplastic agent which has antineoplastic activity against the neoplasm, wherein said antineoplastic agent is or comprises one or more compounds identified in any of steps (vi) or (vii).

21. The method of claim 1, wherein the compounds of the selection of compounds are approved for one or more pharmaceutical purposes.

22. The method of claim 21, wherein the compounds of the selection of compounds are approved for one or more pharmaceutical purposes other than antineoplastic activity and are not approved as antineoplastic agents.

23. A pharmaceutical composition comprising one or more compounds identified in any of steps (vi) or (vii) of claim 19 and a pharmaceutically acceptable carrier.

24. A method for treating a neoplasm in an individual, comprising administering a compound identified in any of steps (vi) or (vii) of claim 19.

25. A computer program comprising instructions which, when the program is executed by a computer, cause the computer to carry out at least steps (iv) and (v) of the method of claim 1.

26. A storage device comprising, stored thereon, the computer program of claim 25.
